(19) United States
(12) Patent Application Publication
(10) Pub. No.: US 2020/0400333 A1
(43) Pub. Date: Dec. 24, 2020

(54) ENVIRONMENT CONTROLLER AND METHOD FOR PREDICTING TEMPERATURE VARIATIONS BASED ON SOUND LEVEL MEASUREMENTS

(71) Applicant: Distech Controls Inc., Brossard (CA)

(72) Inventor: Francois GERVAIS, Lachine (CA)

(21) Appl. No.: 16/445,718

(22) Filed: Jun. 19, 2019

(51) Int. Cl. F24F 11/62; G06N 3/08; G06F 3/16; G01N 33/00

(52) U.S. Cl. CPC F24F 11/62; G01N 33/004; G06F 3/16; G06N 3/08

(57) ABSTRACT

Method and environment controller predicting temperature variations based on sound level measurements. The environment controller determines N consecutive sets of frequency domain sound level measurements. Each set of frequency domain sound level measurements comprises a given number M of sound level amplitudes at the corresponding given number M of frequencies. The environment controller executes a neural network inference engine using a predictive model for inferring one or more output based on inputs. The inputs comprise the N consecutive sets of frequency domain sound level measurements. The one or more output comprises a predicted variation of a temperature. For example, the environment controller receives a plurality of consecutive time domain sound level measurements from a sound sensor and generates the N consecutive sets of frequency domain sound level measurements based on the plurality of consecutive time domain sound level measurements (for instance by using a Fast Fourier Transform algorithm).

### TECHNICAL FIELD

The present disclosure relates to the field of building automation, and more precisely the control of environmental conditions in an area of a building. More specifically, the present disclosure presents an environment controller and a method for predicting temperature variations based on sound level measurements.

### BACKGROUND

Systems for controlling environmental conditions, for example in buildings, are becoming increasingly sophisticated. An environment control system may at once control heating and cooling, monitor air quality, detect hazardous conditions such as fire, carbon monoxide release, intrusion, and the like. Such environment control systems generally include at least one environment controller, which receives measured environmental values, generally from external sensors, and in turn determines set-points or command parameters to be sent to controlled appliances.

The environment controller and the devices under its control (sensors, controlled appliances, etc.) are generally referred to as Environment Control Devices (ECDs). An ECD comprises processing capabilities for processing data received via one or more communication interface and/or generating data transmitted via the one or more communication interface.

For example, the environment controller controls a heating, ventilating, and/or air-conditioning (HVAC) appliance, in order to regulate the temperature, humidity level and CO2 level in an area of a building. The temperature and CO2 level in the area depend on the number of persons present in the area. If the number of persons present in the area increases, the temperature and CO2 level in the area are likely to increase. Similarly, If the number of persons present in the area decreases, the temperature and CO2 level in the area are likely to decrease. By anticipating the variations of temperature and CO2 level in the area, the environment controller is capable of smoothly adjusting the operations of the HVAC appliance, to maintain a safe and comfortable environment for the persons present in the area.

The variations of temperature and CO2 level in the area are correlated to the variations in the number of persons present in the area. However, it is not always possible to directly track the number of persons present in an area. In this case, the evolution of the sound level in the area can be used as a proxy of the evolution of the number of persons present in the area.

Therefore, there is a need for an environment controller and a method for predicting temperature variations based on sound level measurements.

### SUMMARY

According to a first aspect, the present disclosure relates to an environment controller. The environment controller comprises at least one communication interface, memory for storing a predictive model, and a processing unit. The processing unit determines N consecutive sets of frequency domain sound level measurements. Each set of frequency domain sound level measurements comprises a given number M of sound level amplitudes at the corresponding given number M of frequencies. N and M are integers. The processing unit executes a neural network inference engine using the predictive model for inferring one or more output based on inputs. The inputs comprise the N consecutive sets of frequency domain sound level measurements. The one or more output comprises a predicted variation of a temperature.

According to a second aspect, the present disclosure relates to a method for predicting temperature variations based on sound level measurements. The method comprises storing a predictive model in a memory of a computing device. The method comprises determining, by a processing unit of the computing device, N consecutive sets of frequency domain sound level measurements. Each set of frequency domain sound level measurements comprises a given number M of sound level amplitudes at the corresponding given number M of frequencies. N and M are integers. The method comprises executing, by the processing unit of the computing device, a neural network inference engine using the predictive model for inferring one or more output based on inputs. The inputs comprise the N consecutive sets of frequency domain sound level measurements. The one or more output comprises a predicted variation of a temperature.

According to a third aspect, the present disclosure relates to a non-transitory computer program product comprising instructions executable by a processing unit of an environment controller. The execution of the instructions by the processing unit of the environment controller provides for predicting temperature variations based on sound level measurements, by implementing the aforementioned method.

In a particular aspect, determining the N consecutive sets of frequency domain sound level measurements comprises receiving a plurality of consecutive time domain sound level measurements and generating the N consecutive sets of frequency domain sound level measurements based on the plurality of consecutive time domain sound level measurements (for instance by using a Fast Fourier Transform algorithm).

### BRIEF DESCRIPTION OF THE DRAWINGS

Embodiments of the disclosure will be described by way of example only with reference to the accompanying drawings, in which:

FIG. 1 illustrates an environment control system comprising an environment controller and a temperature sensor;

FIGS. 2A and 2B illustrate a method for predicting temperature variations based on sound level measurements;

FIGS. 3A, 3B, 3C and 3D illustrate time domain sound level measurements and corresponding frequency domain sound level measurements;

FIG. 4A is a schematic representations of a neural network inference engine executed by the environment controller of FIG. 1 according to the method of FIGS. 2A-B;

FIG. 4B is a detailed representation of a neural network comprising a 1D convolutional layer;

FIG. 4C is a detailed representation of a neural network comprising a 2D convolutional layer; and

FIG. 5 represents an environment control system where several environment controllers implementing the method illustrated in FIGS. 2A-B are deployed.

### DETAILED DESCRIPTION

The foregoing and other features will become more apparent upon reading of the following non-restrictive description of illustrative embodiments thereof, given by way of example only with reference to the accompanying drawings.

Various aspects of the present disclosure generally address one or more of the problems related to environment control systems for buildings. More particularly, the present disclosure aims at providing solutions for predicting the evolution of an environmental condition (e.g. temperature or CO2 level) in an area of a building in relation to the evolution of the number of persons present in the area. For this purpose, the evolution of the sound level in the area is used as a proxy of the evolution of the number of persons in the area. A neural network is used in this context.

The following terminology is used throughout the present specification:

Environment: condition(s) (temperature, pressure, oxygen level, light level, security, etc.) prevailing in a controlled area or place, such as for example in a building.

Environment control system: a set of components which collaborate for monitoring and controlling an environment.

Environmental data: any data (e.g. information, commands) related to an environment that may be exchanged between components of an environment control system.

Environment control device (ECD): generic name for a component of an environment control system. An ECD may consist of an environment controller, a sensor, a controlled appliance, etc.

Environment controller: device capable of receiving information related to an environment and sending commands based on such information.

Environmental characteristic: measurable, quantifiable or verifiable property of an environment (a building). The environmental characteristic comprises any of the following: temperature, pressure, humidity, lighting, CO2, flow, radiation, water level, speed, sound; a variation of at least one of the following, temperature, pressure, humidity and lighting, CO2 levels, flows, radiations, water levels, speed, sound levels, etc., and/or a combination thereof.

Environmental characteristic value: numerical, qualitative or verifiable representation of an environmental characteristic.

Sensor: device that detects an environmental characteristic and provides a numerical, quantitative or verifiable representation thereof. The numerical, quantitative or verifiable representation may be sent to an environment controller.

Controlled appliance: device that receives a command and executes the command. The command may be received from an environment controller.

Environmental state: a current condition of an environment based on an environmental characteristic, each environmental state may comprise a range of values or verifiable representation for the corresponding environmental characteristic.

VAV appliance: a Variable Air Volume appliance is a type of heating, ventilating, and/or air-conditioning (HVAC) system. By contrast to a Constant Air Volume (CAV) appliance, which supplies a constant airflow at a variable temperature, a VAV appliance varies the airflow at a constant temperature.

Area of a building: the expression ‘area of a building’ is used throughout the present specification to refer to the interior of a whole building or a portion of the interior of the building such as, without limitation: a floor, a room, an aisle, etc.

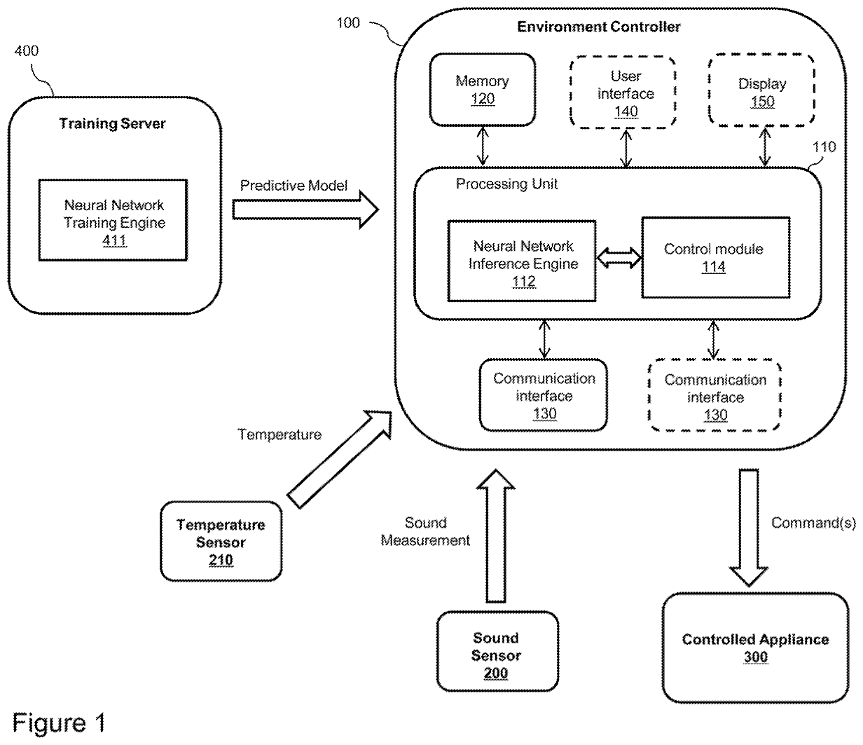

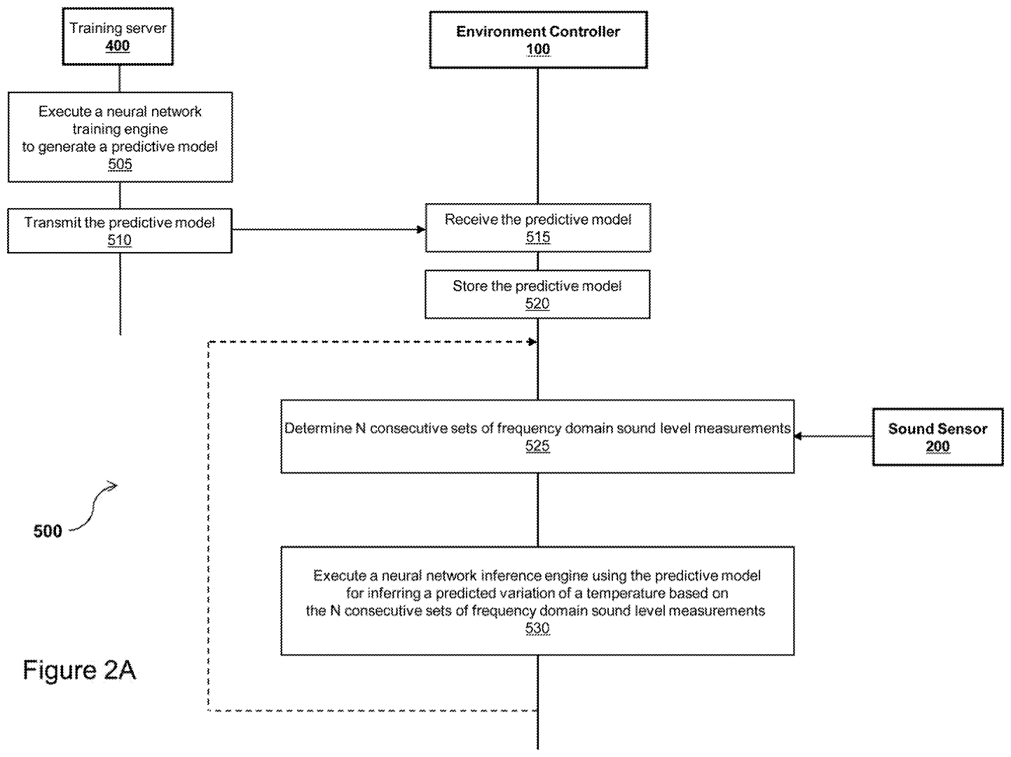

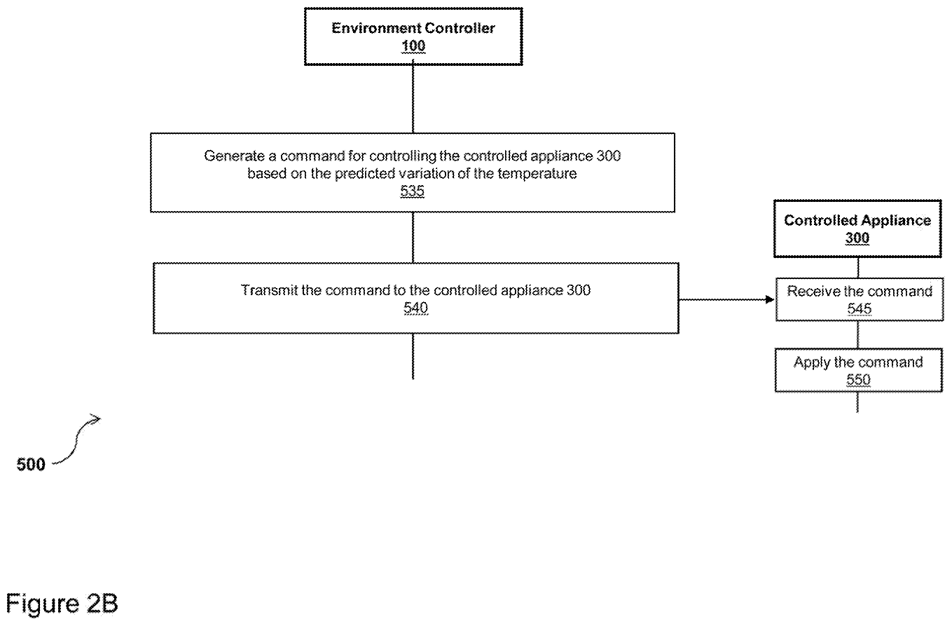

Referring now concurrently to FIGS. 1, 2A and 2B, an environment controller 100 and a method 500 for predicting temperature variations based on sound level measurements are illustrated.

FIG. 1 represents an environment control system where an environment controller 100 exchanges data with other environment control devices (ECDs). The environment controller 100 is responsible for controlling the environment of an area of a building. The environment controller 100 receives from sensors (e.g. 200 and/or 210) environmental characteristic values measured by the sensors. The environment controller 100 generates commands based on the received environmental characteristic values. The generated commands are transmitted to controlled appliances 300 (to control the operations of the controlled appliances 300).

The area under the control of the environment controller 100 is not represented in the Figures for simplification purposes. As mentioned previously, the area may consist of a room, a floor, an aisle, etc. However, any type of area located inside any type of building is considered within the scope of the present disclosure.

In the context of the present disclosure, a particular type of sensor is used: the sound sensor 200, which measures a sound level in the area and transmits the measured sound level to the environment controller 100.

Other examples of sensors include the temperature sensor 210 (measuring a temperature in the area and transmitting the measured temperature to the environment controller 100). Still other examples of sensors not represented in FIG. 1 include a CO2 sensor (measuring a CO2 level in the area and transmitting the measured CO2 level to the environment controller 100), a humidity sensor (measuring a humidity level in the area and transmitting the measured humidity level to the environment controller 100), a lighting sensor (measuring a light level in the area and transmitting the measured light level to the environment controller 100), an occupancy sensor (determining an occupancy of the area and transmitting the determined occupancy to the environment controller 100), etc. Furthermore, each environmental characteristic value measured by a sensor may consist of either a single value (e.g. the current temperature is 24.5 degrees Celsius), or a range of values (e.g. the current temperature is in the range of 24 to 25 degrees Celsius).

In a first implementation, a single sensor measures a given type of environmental characteristic value (e.g. temperature) for the whole area. In a second implementation, the area is divided into a plurality of zones, and a plurality of sensors measures the given type of environmental characteristic value (e.g. temperature) in the corresponding plurality of zones. In the second implementation, the environment controller 100 calculates an average environmental characteristic value in the area (e.g. an average temperature in the area) based on the environmental characteristic values transmitted by the plurality of sensors respectively located in the plurality of zones of the area.

Additional sensor(s) may be deployed outside of the area and report their measurement(s) to the environment controller 100. For example, the area is a room of a building. An external temperature sensor measures an external temperature outside the building and transmits the measured external temperature to the environment controller 100. Similarly, an external humidity sensor measures an external humidity level outside the building and transmits the measured external humidity level to the environment controller 100.

Each controlled appliance 300 comprises at least one actuation module, to control the operations of the controlled appliance 300 based on the commands received from the environment controller 100. The actuation module can be of one of the following type: mechanical, pneumatic, hydraulic, electrical, electronical, a combination thereof, etc. The commands control operations of the at least one actuation module. Although a single controlled appliance 300 is represented in FIG. 1 for simplification purposes, the environment controller 100 may be interacting with a plurality of controlled appliances 300.

An example of a controlled appliance 300 consists of a VAV appliance. Examples of commands transmitted to the VAV appliance 300 include commands directed to one of the following: an actuation module controlling the speed of a fan, an actuation module controlling the pressure generated by a compressor, an actuation module controlling a valve defining the rate of an airflow, etc. This example is for illustration purposes only. Other types of controlled appliances 300 could be used in the context of an environment control system managed by the environment controller 100.

Details of the environment controller 100, sound sensor 200 and control appliance 300 will now be provided.

The environment controller 100 comprises a processing unit 110, memory 120, and a communication interface 130. The environment controller 100 may comprise additional components, such as another communication interface 130, a user interface 140, a display 150, etc.

The processing unit 110 comprises one or more processors (not represented in the Figures) capable of executing instructions of a computer program. Each processor may further comprise one or several cores. The processing unit 110 executes a neural network inference engine 112 and a control module 114, as will be detailed later in the description.

The memory 120 stores instructions of computer program(s) executed by the processing unit 110, data generated by the execution of the computer program(s), data received via the communication interface 130 (or another communication interface), etc. Only a single memory 120 is represented in FIG. 1, but the environment controller 100 may comprise several types of memories, including volatile memory (such as a volatile Random Access Memory (RAM), etc.) and non-volatile memory (such as a hard drive, electrically-erasable programmable read-only memory (EEPROM), flash, etc.).

The communication interface 130 allows the environment controller 100 to exchange data with remote devices (e.g. the sound sensor 200, the controlled appliance 300, etc.) over a communication network (not represented in FIG. 1 for simplification purposes). For example, the communication network is a wired communication network, such as an Ethernet network; and the communication interface 130 is adapted to support communication protocols used to exchange data over the Ethernet network. Other types of wired communication networks may also be supported by the communication interface 130. In another example, the communication network is a wireless communication network, such as a Wi-Fi network; and the communication interface 130 is adapted to support communication protocols used to exchange data over the Wi-Fi network. Other types of wireless communication network may also be supported by the communication interface 130, such as a wireless mesh network, Bluetooth®, Bluetooth® Low Energy (BLE), etc. In still another example, the environment controller 100 comprises two communication interfaces 130. The environment controller 100 communicates with the sensor 200 and the controlled appliance 300 via a first communication interface 130 (e.g. a Wi-Fi interface); and communicates with other devices (e.g. a training server 400) via a second communication interface 130 (e.g. an Ethernet interface). Each communication interface 130 usually comprises a combination of hardware and software executed by the hardware, for implementing the communication functionalities of the communication interface 130

A detailed representation of the components of the sound sensor 200 is not provided in FIG. 1 for simplification purposes. The sound sensor 200 comprises at least one sensing module (e.g. a microphone, a piezoelectric transducer, etc.) for detecting an environmental characteristic (sound). The sound sensor 200 further comprises a communication interface for transmitting to the environment controller 100 an environmental characteristic value (sound level) corresponding to the detected environmental characteristic (sound). The environmental characteristic value is transmitted over a communication network and received via the communication interface 130 of the environment controller 100. The sensor 200 may also comprise a processing unit for generating the environmental characteristic value (sound level) based on the detected environmental characteristic (sound). Examples of a sound level which can be measured by the sound sensor 200 include a sound pressure (also referred to as acoustic pressure) expressed in Pascal (Pa), a sound pressure level expressed in decibels (dB), a sound power (also referred to as acoustic power) expressed in watts (W), etc. The other types of sensors mentioned previously (e.g. temperature sensor 210) generally include the same types of components as those mentioned for the sound sensor 200.

A detailed representation of the components of the controlled appliance 300 is not provided in FIG. 1 for simplification purposes. As mentioned previously, the controlled appliance 300 comprises at least one actuation module. The controlled appliance 300 further comprises a communication interface for receiving one or more commands from the environment controller 100. The one or more commands control operations of the at least one actuation module. The one or more commands are transmitted over a communication network via the communication interface 130 of the environment controller 100. The controlled appliance 300 may also comprise a processing unit for controlling the operations of the at least one actuation module based on the received one or more commands.

A detailed representation of the components of the training server 400 is not provided in FIG. 1 for simplification purposes. The training server 400 comprises a processing unit, memory and a communication interface. The processing unit of the training server 400 executes a neural network training engine 411.

The execution of the neural network training engine 411 generates a predictive model, which is transmitted to the environment controller 100 via the communication interface of the training server 400. The predictive model is transmitted over a communication network and received via the communication interface 130 of the environment controller 100.

Reference is now made concurrently to FIGS. 1, 2A, 2B, 3A, 3B, 3C and 3D. At least some of the steps of the method 500 represented in FIGS. 2A and 2B are implemented by the environment controller 100, for predicting temperature variations based on sound level measurements. However, the present disclosure is not limited to the environment controller 100, but is applicable to any type of computing device capable of implementing the steps of the method 500.

A dedicated computer program has instructions for implementing at least some of the steps of the method 500. The instructions are comprised in a non-transitory computer program product (e.g. the memory 120) of the environment controller 100. The instructions provide for predicting temperature variations based on sound level measurements, when executed by the processing unit 110 of the environment controller 100. The instructions are deliverable to the environment controller 100 via an electronically-readable media such as a storage media (e.g. CD-ROM, USB key, etc.), or via communication links (e.g. via a communication network through the communication interface 130).

The instructions of the dedicated computer program executed by the processing unit 110 implement the neural network inference engine 112 and the control module 114. The neural network inference engine 112 provides functionalities of a neural network, allowing to infer output(s) based on inputs using the predictive model, as is well known in the art. The control module 114 provides functionalities allowing the environment controller 100 to interact with and control other devices (e.g. the sound sensor 200 and the controlled appliance 300).

The method 500 comprises the step 505 of executing the neural network training engine 411 to generate the predictive model. Step 505 is performed by the processing unit of the training server 400. This step will be further detailed later in the description.

The method 500 comprises the step 510 of transmitting the predictive model generated at step 505 to the environment controller 100, via the communication interface of the training server 400. Step 510 is performed by the processing unit of the training server 400.

The method 500 comprises the step 515 of receiving the predictive model from the training server 400, via the communication interface 130 of the environment controller 100. Step 515 is performed by the processing unit 110 of the environment controller 100.

The method 500 comprises the step 520 of storing the predictive model in the memory 120 of the environment controller 100. Step 520 is performed by the processing unit 110 of the environment controller 100.

The method 500 comprises the step 525 of determining N consecutive sets of frequency domain sound level measurements Step 525 is performed by the control module 114 executed by the processing unit 110. Each set of frequency domain sound level measurements comprises a given number M of sound level amplitudes at the corresponding given number M of frequencies. N and M are integers.

In a first exemplary implementation, the determination performed at step 525 comprises receiving a plurality of consecutive time domain sound level measurements for an area, via the communication interface 130. The plurality of consecutive time domain sound level measurements are measured by the sound sensor 200 and transmitted to the environment controller 100.

The sound sensor 200 is located in the area for which the time domain sound level measurements are performed. As mentioned previously, examples of areas include a room of a building, an aisle of the building, a floor of the building etc. Alternatively, the plurality of consecutive time domain sound level measurements are not transmitted directly from the sound sensor 200 to the environment controller 100, but transit through one or more intermediate device.

The determination performed at step 525 further comprises generating the N consecutive sets of frequency domain sound level measurements based on the plurality of consecutive time domain sound level measurements received from the sound sensor 200.

As mentioned previously, each sound level measurement of the time domain sound level measurements consists of one of the following: a sound pressure, a sound pressure level, a sound power, etc. Each sound level amplitude of the frequency domain sound level measurements consists of one of the corresponding following: a sound pressure at a given frequency, a sound pressure level at a given frequency, a sound power at a given frequency, etc.

For example, the determination of the N consecutive sets of frequency domain sound level measurements based on the plurality of consecutive time domain sound level measurements uses a Fast Fourier Transform (FFT) algorithm. The FFT algorithm is well known in the art for transforming a time domain signal into a frequency domain signal. It is an adaptation of the Discrete Fourier Transform (DFT) algorithm, which is also well known in the art.

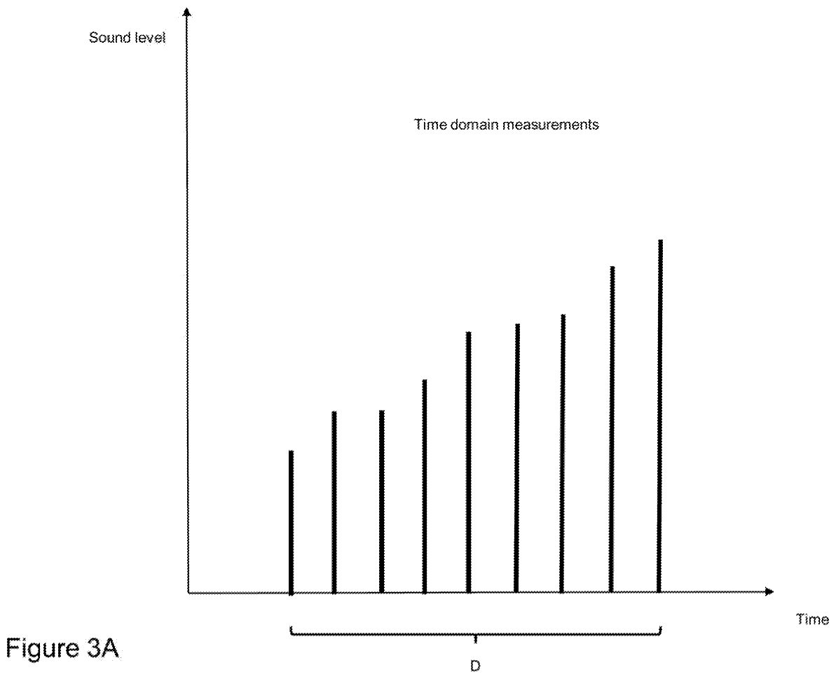

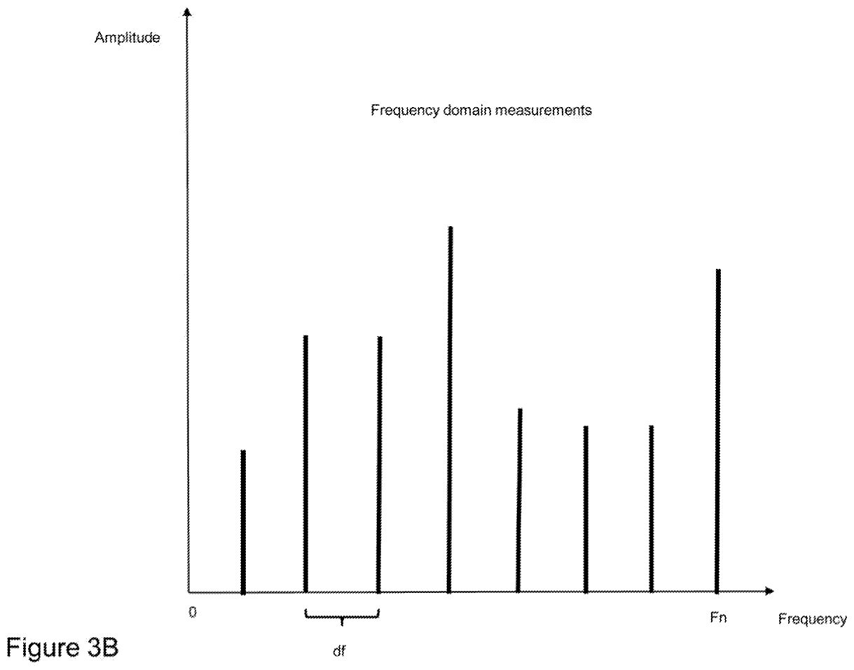

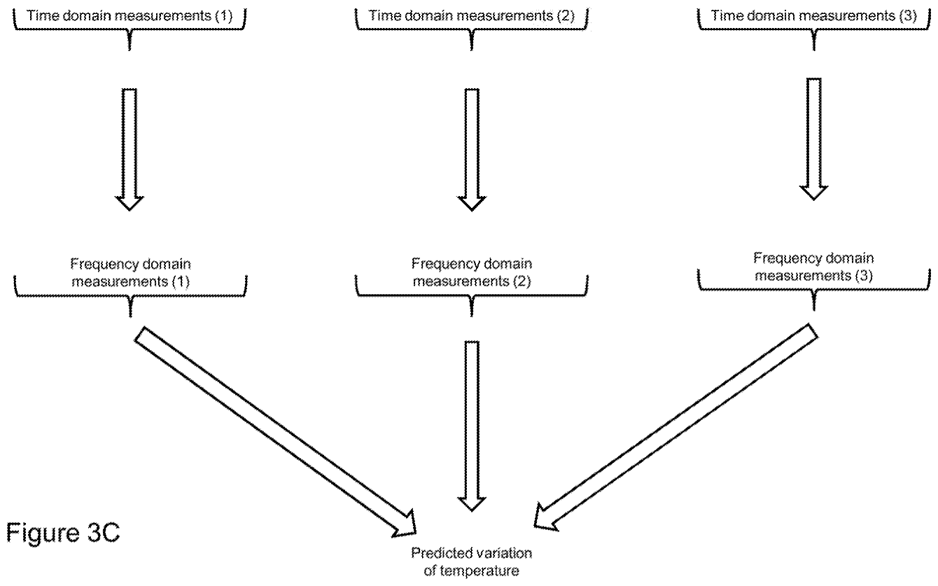

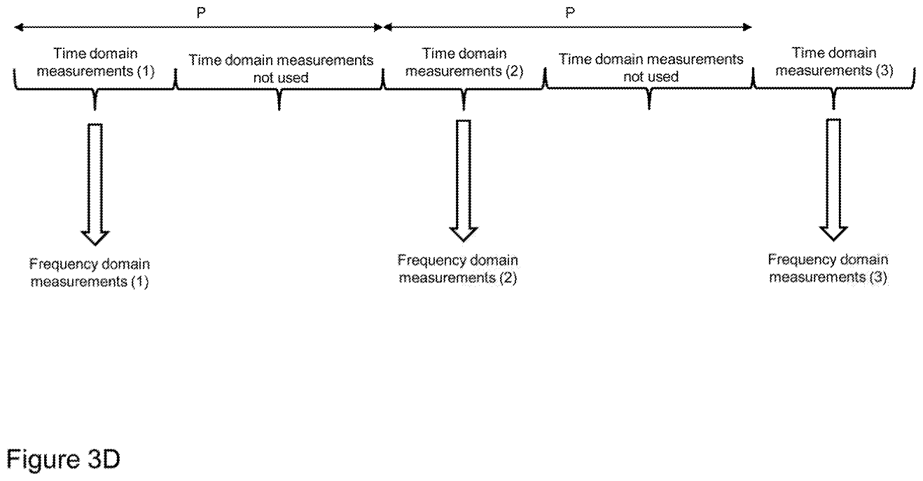

The FFT algorithm operates with two parameters. The first parameter is the sampling rate or sampling frequency Fₛ, which defines the average number of time domain sound level measurements per second. For example, Fₛ is equal to 48000 time domain measurements per second (48 kilohertz or kHz). For illustration purposes only, FIG. 3A illustrates an exemplary plurality of consecutive time domain sound level measurements.

The second parameter is the selected number of samples or blocklength BL, which is always an integer power to the base 2 in the FFT. For example, BL is equal to 1024 samples.

The following parameters of the FFT are deducted from Fₛ and BL. The bandwidth Fₙ=Fₛ/2 indicates the theoretical maximum frequency that can be determined by the FFT. In the current example, Fₙ is equal to 24 kHz. The measurement duration D=BL/Fₛ. In the current example, D is equal to 1024/48000=21.3 milliseconds. The frequency resolution df=Fₛ/BL indicates the frequency spacing between two measurement results. In the current example, df is equal to 48000/1024=46.88 Hz.

For illustration purposes only, FIG. 3B illustrates an exemplary set of consecutive frequency domain sound level measurements corresponding to the FFT of the plurality of consecutive time domain sound level measurements of FIG. 3A. The measurement duration D is represented in FIG. 3A. The frequency resolution df and the bandwidth Fₙ are represented in FIG. 3B.

The sampling frequency Fₛ is determined by the capabilities of the sound sensor 200, since the sound sensor 200 is the equipment which performs the consecutive sound level measurements in the time domain. The blocklength BL can be adjusted accordingly, for example to reach a target value for the measurement duration D and/or frequency resolution df. For instance, increasing the blocklength BL for a given sampling frequency Fₛ increases the measurement duration D and decreases the frequency resolution df.

FIG. 3C illustrates an exemplary implementation where the number N of consecutive sets of frequency domain sound level measurements determined at step 525 is 3 (N=3). However, as will be illustrated later in the description, higher values (e.g. N=10 or N=20) can be used as well.

A first plurality of samples of consecutive time domain sound level measurements is used for generating a first set of frequency domain sound level measurements by applying the FFT.

A second plurality of samples of consecutive time domain sound level measurements is used for generating a second set of frequency domain sound level measurements by applying the FFT.

A third plurality of samples of consecutive time domain sound level measurements is used for generating a third set of frequency domain sound level measurements by applying the FFT.

The first, second and third sets of frequency domain sound level measurements are used for the inputs of the neural network inference engine 112 to infer a predicted variation of a temperature in the area where the sound sensor 200 is deployed.

In the previous example, the measurement duration D is equal to 21.3 milliseconds. Thus, a new set of frequency domain sound level measurements is generated approximately every 21.3 milliseconds. Consequently, step 530 is repeated approximately every D*N=21.3*3=64 milliseconds.

Alternatively, a new set of frequency domain sound level measurements is generated periodically at a configured period P. For example, the period P is set to 1 second. At a time t, a first set of frequency domain sound level measurements is generated. At a time t+P (e.g. t+1 second), a second set of frequency domain sound level measurements is generated. At a time t+2*P (e.g. t+2 second), a third set of frequency domain sound level measurements is generated. The three generated sets are then used at step 530. In this case, step 530 is repeated every P*N=1*3=3 seconds. Thus, a predicted variation of a temperature is calculated every 3 seconds. FIG. 3D illustrates this implementation (FIG. 3D is for illustration purposes only and the time scales are not intended to be accurate). Between two consecutive periods where the sets of frequency domain sound level measurements are generated, the time domain sound level measurements received from the sensor 200 are not used. If possible, the sensor 200 may be configured to send the appropriate number of time domain sound level measurements (for applying the FFT to generate one set of frequency domain sound level measurements) at the configured period P (e.g. every 1 second). The length of the period P is determined experimentally and may be equal to one or more second, one or more minute, etc.

In the previous example, Fₙ is equal to 24 kHz and df is equal to 48000/1024=46.88 Hz. Thus, each set of frequency domain sound level measurements comprises approximately Fₙ/df=24000/46.88=512 measurements (each measurement corresponds to an amplitude of the sound level at a given frequency). If the number of measurements is too high to be used as inputs of the neural network inference engine 112 at step 530, a subset of the measurement is selected for being used at step 530. For example, only 20 measurements among the 512 measurements are selected for each set of frequency domain sound level measurements. Thus, with N=3, the number of values used for the inputs of the neural network inference engine 112 at step 530 is 20*3=60 values. The selection of the 20 measurements may be random. Alternatively, 20 frequencies are selected via an experimental process consisting in testing the accuracy of the output of the neural network inference engine 112 for various candidate frequencies. Alternatively, a basic sampling mechanism is used, which consists in selecting one measurements every 26 (approximately 512/20) measurements of the 512 measurements of a given set.

In another exemplary implementation, the determination performed at step 525 comprises directly receiving the N consecutive sets of frequency domain sound level measurements via the communication interface 130.

For example, the sound sensor 200 has the capability of generating the sets of frequency domain sound level measurements (e.g. by applying the previously described FFT to time domain sound level measurements collected by the sound sensor 200). The frequency domain sound level measurements are transmitted by the sensor 200 to the environment controller 100.

In another example, an intermediate computing device (not represented in the Figures) has the capability of generating the sets of frequency domain sound level measurements based on data received from the sound sensor 200 (e.g. based on a plurality of time domain sound level measurement). The intermediate computing device transmits the generated sets of frequency domain sound level measurements to the environment controller 100.

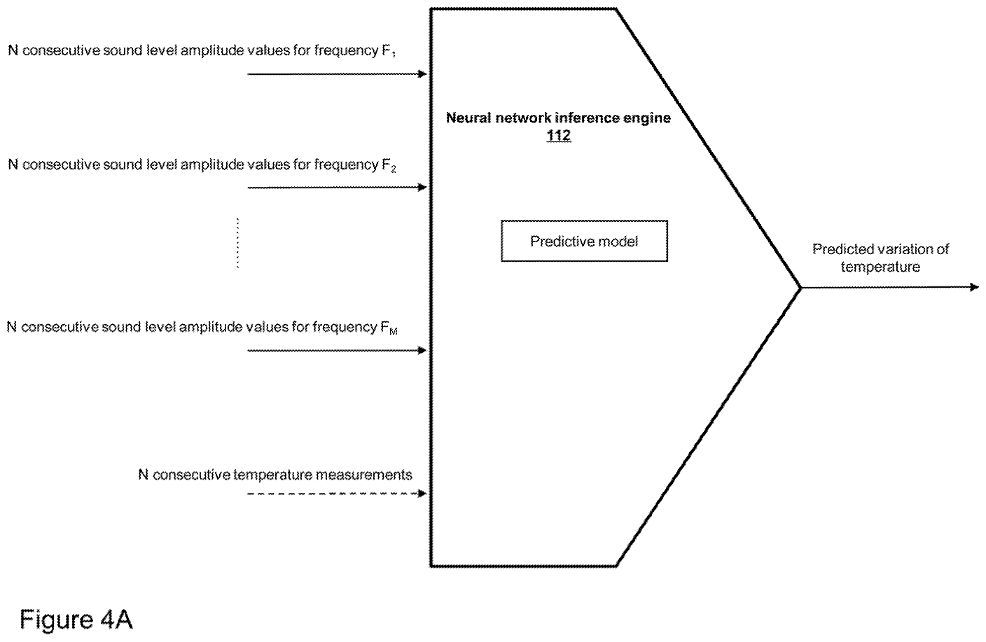

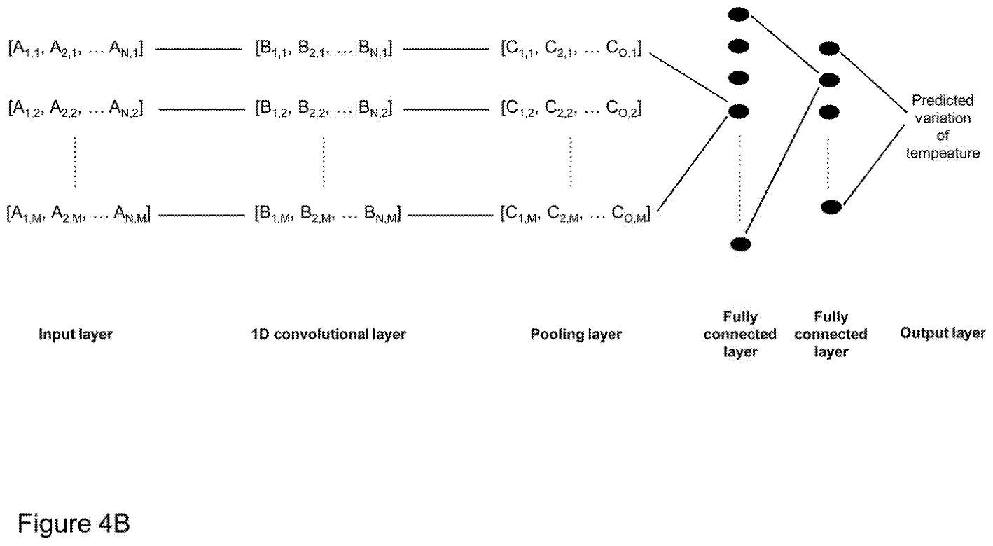

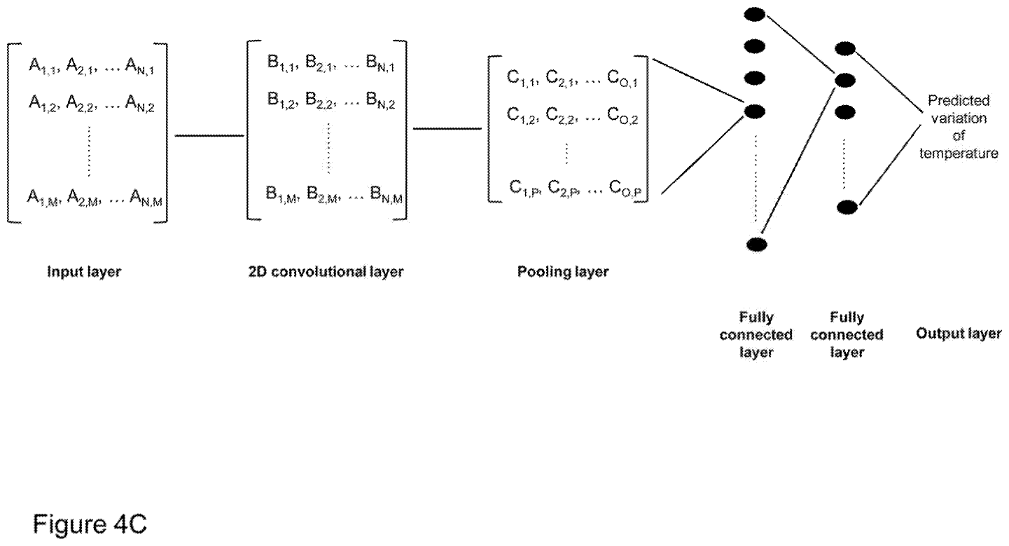

The method 500 comprises the step 530 of executing the neural network inference engine 112 using the predictive model (stored at step 520) for inferring a predicted variation of a temperature in the area, based on the N consecutive sets of frequency domain sound level measurements (determined at step 525). Each one of the N (e.g. 3) consecutive sets of frequency domain sound level measurements respectively includes the aforementioned given number M (e.g. 20) of sound level amplitudes at the corresponding given number M of frequencies. Step 530 is performed by the processing unit 110 of the environment controller 100. Details of how the N consecutive sets of frequency domain sound level measurements are used to generate the inputs of the neural network inference engine 112 will be provided later in the description, in relation to FIGS. 4A, 4B and 4C.

The inputs used by the neural network inference engine 112 may include other parameter(s), in addition to the N consecutive sets of frequency domain sound level measurements. Similarly, the outputs generated by the neural network inference engine 112 may include other predicted data, in addition to the predicted variation of a temperature in the area.

For example, the inputs further include N consecutive temperature measurements corresponding to the N consecutive sets of frequency domain sound level measurements. The method 500 comprises the optional step (not represented in FIG. 2A for simplification purposes) of determining the N consecutive temperature measurements, which is performed in parallel to step 525. At step 530, the neural network inference engine 112 uses the N consecutive sets of frequency domain sound level measurements and the corresponding N consecutive temperature measurements as inputs.

The determination of each consecutive temperature measurement can be implemented in different ways. We consider a reference interval of time, during which a given set of frequency domain sound level measurements is determined. During the reference interval of time, the temperature sensor 210 is configured to make a single temperature measurement, which is transmitted to the environment controller 100 and used as input (at step 530) for the reference interval of time. Alternatively, during the reference interval of time, the temperature sensor 210 is configured to make several temperature measurements, the average of the several temperature measurements being calculated and transmitted by the temperature sensor 210 to the environment controller 100, and the calculated average is used as input (at step 530) for the reference interval of time. In still another alternative implementation, the temperature sensor 210 has no knowledge of the reference interval of time and simply transmits temperature measurements to the environment controller 100. In this case, for each reference interval of time, the environment controller 100 sends a request to the temperature sensor 210 to transmit a temperature measurement. The temperature sensor 210 sends the requested temperature measurement to the environment controller 100, which is used as input (at step 530) for the reference interval of time. Instead of a single temperature measurement for each reference interval of time, the environment controller 100 may request and receive a plurality of temperature measurements from the temperature sensor 210. The environment controller 100 uses as input (at step 530) the average of the plurality of received temperature measurements for the reference interval of time. In yet another alternative implementation, during each reference interval of time, the temperature sensor 210 spontaneously transmits one or more temperature measurement to the environment controller 100. The environment controller 100 uses for the reference interval of time as input (at step 530) a single one of the temperature measurement(s) received from the temperature sensor 210 or an average of several temperature measurements received from the temperature sensor 210. The predictive model has been trained at step 505 to use N consecutive temperature measurements as inputs (corresponding to the N consecutive sets of frequency domain sound level measurements).

Alternatively, a single temperature measurement is used as input at step 530. The single temperature measurement is determined during a reference interval of time, during which the N (e.g. 3) sets of frequency domain sound level measurements are determined. The determination of the single temperature measurement (based on measurements made by the temperature sensor 210) is made in a manner similar to one of the previously mentioned alternatives.

In another example, the outputs further include a predicted variation of CO2 level in the area. The predictive model has been trained at step 505 to predict both a variation of temperature and a variation of CO2 level in the area.

In this case, the inputs may further include N consecutive CO2 level measurements corresponding to the N consecutive sets of frequency domain sound level measurements. The determination of the N consecutive CO2 level measurements is similar to the aforementioned determination of the N consecutive temperature measurements, and uses a CO2 sensor (not represented in the Figures) located in the area. The predictive model has been trained at step 505 to further use N consecutive CO2 level measurements as inputs. As mentioned previously in relation to the temperature measurement, a single CO2 level measurement corresponding to the N consecutive sets of frequency domain sound level measurements may also be used as input.

The method 500 comprises the step 535 of generating a command for controlling the controlled appliance 300 based on the predicted variation of the temperature in the area. Step 535 is performed by the control module 114 executed by the processing unit 110. Optionally, the generation of the command uses one or more additional parameter, such as a current temperature in the area transmitted by the temperature sensor 210.

As mentioned previously, an example of controlled appliance 300 is a VAV appliance. Examples of commands for controlling the VAV appliance 300 include commands directed to one of the following actuation modules of the VAV appliance 300: an actuation module controlling the speed of a fan, an actuation module controlling the pressure generated by a compressor, an actuation module controlling a valve defining the rate of an airflow, etc.

In an alternative implementation, steps 525-530 are repeated several times before performing step 535. The command generated at step 535 is based on several consecutive predicted variations of the temperature in the area (generated by the repetition of step 525).

The method 500 comprises the step 540 of transmitting the command (generated at step 535) to the controlled appliance 300 via the communication interface 130. Step 540 is performed by the control module 114 executed by the processing unit 110.

The method 500 comprises the step 545 of receiving the command at the controlled appliance 300, via the communication interface of the controlled appliance 300. Step 545 is performed by the processing unit of the controlled appliance 300.

The method 500 comprises the step 550 of applying the command at the controlled appliance 300. Step 550 is performed by the processing unit of the controlled appliance 300. Applying the command consists in controlling one or more actuation module of the controlled appliance 300 based on the received command.

In an alternative implementation, instead of performing steps 535 and 540, the environment controller 100 transmits (via the communication interface 130) the predicted variation of the temperature in the area to another device (not represented in the Figures). The other device performs steps 535 and 540 instead of the environment controller 100.

A plurality of commands may be generated at step 535 and transmitted at step 540 to the same controlled appliance 300. Alternatively, the same command may be generated at step 535 and transmitted at step 540 to a plurality of controlled appliances 300. In yet another alternative, a plurality of commands may be generated at step 535 and transmitted at step 540 to a plurality of controlled appliances 300. In any case, the command(s) are always based on the predicted variation of the temperature (inferred at step 530).

Various algorithms may be used for generating the command based on one or more parameter comprising the predicted variation of the temperature in the area. These algorithms are out of the scope of the present disclosure. However, examples of such algorithms are well known in the art of environment control.

If the one or more output generated at step 530 further comprises a predicted variation of CO2 level in the area, the generation of a particular command may use the predicted variation of temperature in the area only, the predicted variation of CO2 level in the area only, or a combination of the predicted variation of temperature in the area and the predicted variation of CO2 level in the area. In this case, the generation of a particular command optionally uses a current CO2 level in the area transmitted by a CO2 sensor located in the area, in addition to the predicted variation of CO2 level in the area.

The steps of the method 500 involving the reception or the transmission of data by the environment controller 100 may use the same communication interface 130 or different communication interfaces 130. For example, step 515 uses a first communication interface 130 of the Ethernet type, while steps 525 and 540 use a second communication interface 130 of the Wi-Fi type. In another example, steps 515, 525 and 540 use the same communication interface 130 of the Wi-Fi type.

FIG. 4A illustrates the inputs and the outputs used by the neural network inference engine 112 when performing step 530.

As mentioned previously, each one of the N consecutive sets of frequency domain sound level measurements determined at step 525 of the method 500 comprises the number M of sound level amplitudes at the corresponding number M of frequencies.

The first set of frequency domain sound level measurements consists of a first set of M sound level amplitude values at respective frequencies F₁, F₂ . . . F_{M}. The second set of frequency domain sound level measurements consists of a second set of M sound level amplitude values at respective frequencies F₁, F₂ . . . F_{M}. The last set (N) of frequency domain sound level measurements consists of a last set of M sound level amplitude values at respective frequencies F₁, F₂ . . . F_{M}.

As illustrated in FIG. 4A, the inputs of the neural network inference engine 112 are grouped by frequencies. The N consecutive sound level amplitude values for frequency F₁ consist of [A_{1,1}, A_{2,1}, . . . A_{N,1}]. The N consecutive sound level amplitude values for frequency F₂ consist of [A_{1,2}, A_{2,2}, . . . A_{N,2}]. The N consecutive sound level amplitude values for frequency F_{M} consist of [A_{1,M}, A_{2,M}, . . . A_{N,M}]. Each sound level amplitude value A_{i,j} consists of the sound level amplitude at frequency Fⱼ for the determined set i of frequency domain sound level measurements, where i varies from 1 to N and j varies from 1 to M.

If the value of N is low (e.g. 3, 4 or 5), the neural network inference engine 112 implements a neural network comprising an input layer, one or more intermediate layer, and an output layer; where all the layers are fully connected. The input layer comprises at least N*M neurons, where each one among the N*M neurons of the input layer receives an amplitude A_{i,j} (i varies from 1 to N and j varies from 1 to M). For example, if N=5 and M=20, the input layer comprises at least 100 neurons respectively receiving the amplitudes A_{i,j}. The output layer comprises at least one neuron outputting the predicted variation of temperature. The generation of the outputs based on the inputs using weights allocated to the neurons of the neural network is well known in the art for a neural network with only fully connected layers. The architecture of the neural network, where each neuron of a layer (except for the first layer) is connected to all the neurons of the previous layer is also well known in the art.

If the value of N is higher (e.g. 10, 20 or 30), the neural network inference engine 112 implements a convolutional neural network comprising an input layer, several intermediate layers, and an output layer. The layers immediately after the input layer comprise one or more convolutional layer. Optionally, each convolutional layer is followed by a pooling layer. The rest of the layers up to the output layer consists of fully connected layers.

In a first implementation, one dimensional (1D) convolution is used. The input layer comprises at least M neurons, where each one among the M neurons of the input layer receives a one-dimension matrix with amplitude values (i varies from 1 to N for each matrix and j varies from 1 to M across the matrices). For example, if N=25 and M=20, the input layer comprises at least 20 neurons respectively receiving the j^{th} matrixes [A_{1,j}, A_{2,j}, . . . A_{25,j}]. Each matrix [A_{1,j}, A_{2,j}, . . . A_{25,j}] represents the evolution of the amplitude at a given frequency Fⱼ over the 25 consecutives sets determined at step 525 of the method 500.

The first layer following the input layer is a 1D convolutional layer applying a 1D convolution to the M matrixes [A_{1,j}, A_{2,j}, . . . A_{N,j}]. The 1D convolution uses a one dimension filter of size S lower than N. The output of the 1D convolutional layer consists in M resulting matrixes [B_{1,j}, B_{2,j}, . . . B_{N,j}]. As mentioned previously, the 1D convolutional layer may be followed by a pooling layer for reducing the size of the M resulting matrixes [B_{1,j}, B_{2,j}, . . . B_{N,1}] into respective reduced matrixes [C_{1,j}, C_{2,j}, . . . C_{O,j}] where O is lower than N. Various algorithms (e.g. maximum value, minimum value, average value, etc.) can be used for implementing the pooling layer, as is well known in the art (a one dimension filter of given size is also used by the pooling layer).

The neural network may include several consecutive 1D convolutional layers, optionally respectively followed by pooling layers. The M matrixes [A_{1,j}, A_{2,j}, . . . A_{N,j}] are processed independently of one another along the chain of 1D convolutional layer(s) and optional pooling layer(s).

The chain of 1D convolutional layer(s) and optional pooling layer(s) is followed by one or more fully connected layer, which operates with weights associated to neurons, as is well known in the art.

FIG. 4B is a schematic exemplary representation of this first implementation, where the neural network comprises the input layer, one 1D convolutional layer, one pooling layer, two fully connected layers and the output layer.

In a second implementation, two dimensional (2D) convolution is used. The input layer comprises at least one neuron receiving a two-dimensions matrix with amplitude values (i varies from 1 to N and j varies from 1 to M). For example, if N=25 and M=20, the input layer comprises a neuron receiving the 25×20 input matrix. Following is a representation of the input matrix:

$$[ {A}_{1 , 1} , {A}_{2 , 1} , … {A}_{N , 1} , {A}_{1 , 2} , {A}_{2 , 2} , … {A}_{2 N , 2} , … {A}_{1 , M} , {A}_{2 , M} , … {A}_{N , M} ]$$

Each line [A_{1,j}, A_{2,j}, . . . A_{N,j}] of the matrix represents the evolution of the amplitude at a given frequency Fⱼ over the N (e.g. 25) consecutives sets determined at step 525 of the method 500.

The first layer following the input layer is a 2D convolutional layer applying a 2D convolution to the N×M (e.g. 25×20) matrix. The 2D convolution uses a two-dimensions filter of size S×T, where S is lower than N and T is lower than M. The output of the 2D convolutional layer consists in a resulting matrix:

$$[ {B}_{1 , 1} , {B}_{2 , 1} , … {B}_{N , 1} , {B}_{1 , 2} , {B}_{2 , 2} , … {B}_{N , 2} , … {B}_{1 , M} , {B}_{2 , M} , … {B}_{N , M} ]$$

As mentioned previously, the 2D convolutional layer may be followed by a pooling layer for reducing the size of the resulting matrix into a reduced matrix:

$$[ {C}_{1 , 1} , {C}_{2 , 1} , … {C}_{O , 1} , {B}_{1 , 2} , {B}_{2 , 2} , … {B}_{O , 2} , … {B}_{1 , P} , {B}_{2 , P} , … {B}_{O , P} ]$$

where O is lower than N and P is lower than M. Various algorithms can be used for implementing the pooling layer, as is well known in the art (a two-dimensions filter of given size is also used by the pooling layer).

The neural network may include several consecutive 2D convolutional layers, optionally respectively followed by pooling layers.

The chain of 2D convolutional layer(s) and optional pooling layer(s) is followed by one or more fully connected layer, which operates with weights associated to neurons, as is well known in the art.

FIG. 4C is a schematic exemplary representation of this second implementation, the neural network comprising the input layer, one 2D convolutional layer, one pooling layer, two fully connected layers and the output layer.

The usage of one or more 1D convolutional layer allows to detect patterns between values of the amplitude at a given frequency, but not across frequencies. The usage of one or more 2D convolutional layer allows to detect patterns between values of the amplitude at different frequencies.

As mentioned previously and optionally, a plurality of N consecutive temperature measurements in the area is also used for the inputs of the neural network inference engine 112 when performing step 530.

In the case where the neural network inference engine 112 implements a neural network only comprising fully connected layers, the input layer comprises N additional neurons for receiving the N consecutive temperature measurements.

In the case where the neural network inference engine 112 implements a neural network comprising 1D convolutional layer(s) and optionally pooling layer(s), the input layer comprises one additional neuron for receiving a matrix comprising the N consecutive temperature measurements. The processing of the matrix comprising the N consecutive temperature measurements is similar to the processing of the M matrixes comprising the amplitude values [A_{i,1}, A_{i,2}, . . . A_{1,N}]. This use case is not represented in FIG. 4B for simplification purposes.

In the case where the neural network inference engine 112 implements a neural network comprising 2D convolutional layer(s) and optionally pooling layer(s), the input layer comprises one additional neuron for receiving a matrix comprising the N consecutive temperature measurements. The matrix comprising the N consecutive temperature measurements is treated by one or more 1D convolutional layer operating in parallel with the one or more 2D convolutional layer treating the amplitudes. Alternatively, one line comprising the N consecutive temperature measurements is added to the matrix comprising the amplitudes, and the temperature measurements are also treated by the one or more 2D convolutional layer. This use case is not represented in FIG. 4C for simplification purposes.

The usage of optional inputs (e.g. the plurality of consecutive temperature measurements in the area) may improve the accuracy and resiliency of the inferences performed by the neural network inference engine 112 (at the cost of complexifying the predictive models used by the neural network inference engine 112). The relevance of using some optional inputs is generally evaluated during the training phase, when the predictive models are generated (and tested) with a set of training (and testing) inputs and outputs dedicated to the training (and testing) phase.

The optional output consisting of the predicted variation of CO2 level and the optional input(s) consisting of the CO2 level measurement(s) are not represented in FIGS. 4A, 4B and 4C for simplification purposes.

Referring back to FIGS. 1 and 2A, the training phase performed by the neural network training engine 411 of the training server 400 (when performing step 505 of the method 500) is well known in the art. The inputs and output(s) of the neural network training engine 411 are the same as those previously described for the neural network inference engine 112. The training phase consists in generating the predictive model that is used during the operational phase by the neural network inference engine 112. The predictive model includes the number of layers, the number of neurons per layer, and the weights associated to the neurons of the fully connected layers. The values of the weights are automatically adjusted during the training phase. Furthermore, during the training phase, the number of layers and the number of neurons per layer can be adjusted to improve the accuracy of the model.

The inputs and output(s) for the training phase of the neural network can be collected through an experimental process. For example, a plurality of combinations of N sets of frequency domain sound level measurements are collected for different combination of persons present in the area, entering the area, leaving the area, etc. The collection is made by deploying the sound level sensor 200 in the area; and further deploying a computing device (e.g. the training server 400) capable of performing the determination of the N sets of frequency domain sound level measurements based on data provided by the sound level sensor 200. For each combination of N sets of frequency domain sound level measurements, a corresponding variation of the temperature in the area is determined using data provided by the temperature sensor 210. The combinations of N sets of frequency domain sound level measurements and the corresponding variations of the temperature in the area are respectively used as inputs and output(s) by the neural network training engine 411 for generating the predictive model by adjusting the weights associated to the neurons of the fully connected layers.

Various techniques well known in the art of neural networks are used for performing (and improving) the generation of the predictive model, such as forward and backward propagation, usage of bias in addition to the weights (bias and weights are generally collectively referred to as weights in the neural network terminology), reinforcement training, etc.

In the case where a convolutional layer is used, parameters of the convolutional layer are also adapted during the training phase. For example, the size of the filter used for the convolution is determined during the training period. The parameters of the convolutional layer are included in the predictive model.

Similarly, in the case where a pooling layer is used, parameters of the pooling layer are also adapted during the training phase. For example, the algorithm and the size of the filter used for the pooling operation are determined during the training period. The parameters of the polling layer are included in the predictive model.

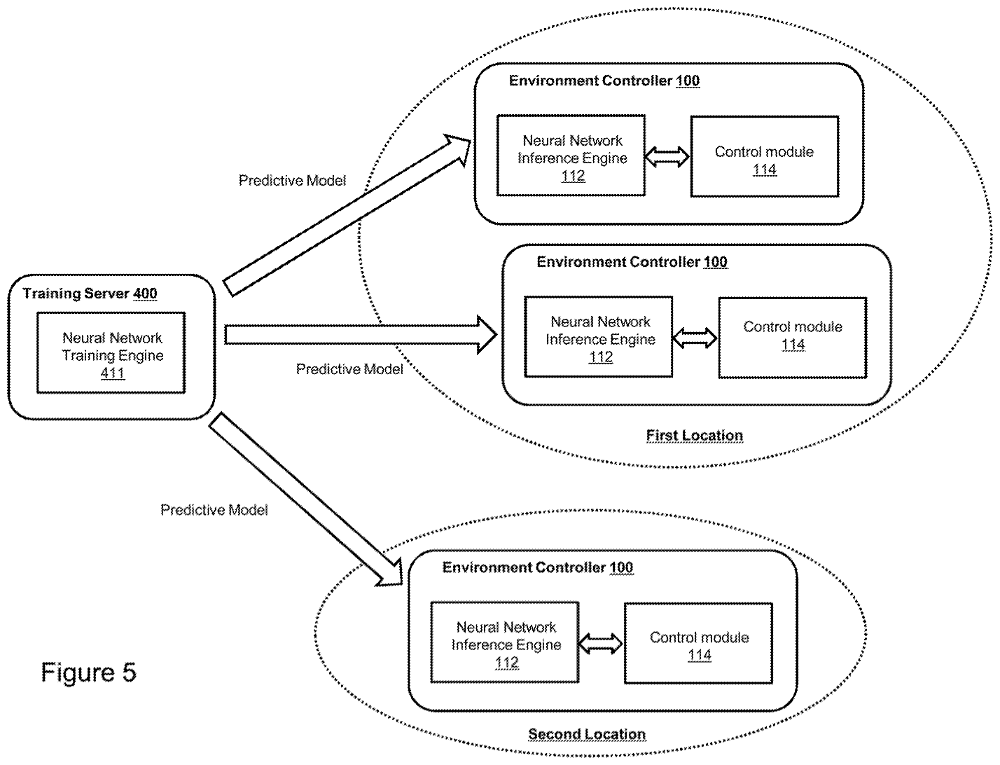

Reference is now made concurrently to FIGS. 1, 2A-B and 5, where FIG. 5 illustrates the usage of the method 500 in a large environment control system.

A first plurality of environment controllers 100 implementing the method 500 are deployed at a first location. Only two environment controllers 100 are represented for illustration purposes, but any number of environment controllers 100 may be deployed.

A second plurality of environment controllers 100 implementing the method 500 are deployed at a second location. Only one environment controller 100 is represented for illustration purposes, but any number of environment controllers 100 may be deployed.

The first and second locations may consist of different buildings, different floors of the same building, etc. Only two locations are represented for illustration purposes, but any number of locations may be considered.

Each environment controller 100 represented in FIG. 5 corresponds to the environment controller 100 represented in FIG. 1, and executes both the control module 114 and the neural network inference engine 112. Each environment controller 100 receives a predictive model from the centralized training server 400 (e.g. a cloud based training server 400 in communication with the environment controllers 100 via a networking infrastructure, as is well known in the art). The same predictive model is used for all the environment controllers 100. Alternatively, a plurality of predictive models is generated, and takes into account specific operating conditions of the environment controllers 100. For example, a first predictive model is generated for the environment controllers 100 controlling a first area having a first set of geometric properties, and a second predictive model is generated for the environment controllers 100 controlling a second area having a second set of geometric properties. Examples of geometric properties include a volume of the area, a surface of the area, a shape of the area, a height of the area, etc.

FIG. 5 illustrates a decentralized architecture, where the environment controllers 100 take autonomous decisions for controlling the controlled appliances 300, using the predictive model as illustrated in the method 500.

In an alternative configuration, at least some of the environment controllers 100 also execute the neural network training engine 411. In this case, the training phase (performed by the neural network training engine 411 for generating the predictive model) and the operational phase (performed by the neural network inference engine 112) are both executed by the environment controller 100.

Although the present disclosure has been described hereinabove by way of non-restrictive, illustrative embodiments thereof, these embodiments may be modified at will within the scope of the appended claims without departing from the spirit and nature of the present disclosure.

What is claimed is:

1. An environment controller comprising:
at least one communication interface;
memory for storing a predictive model; and
a processing unit for:
determining N consecutive sets of frequency domain sound level measurements, each set of frequency domain sound level measurements comprising a given number M of sound level amplitudes at the corresponding given number M of frequencies, N and M being integers; and
executing a neural network inference engine using the predictive model for inferring one or more output based on inputs, the inputs comprising the N consecutive sets of frequency domain sound level measurements, the one or more output comprising a predicted variation of a temperature.

2. The environment controller of claim 1, wherein determining the N consecutive sets of frequency domain sound level measurements comprises receiving the N consecutive sets of frequency domain sound level measurements via the at least one communication interface.

3. The environment controller of claim 2, wherein the N consecutive sets of frequency domain sound level measurements are received from a sound sensor.

4. The environment controller of claim 1, wherein determining the N consecutive sets of frequency domain sound level measurements comprises:
receiving a plurality of consecutive time domain sound level measurements via the at least one communication interface; and
generating the N consecutive sets of frequency domain sound level measurements based on the plurality of consecutive time domain sound level measurements.

5. The environment controller of claim 4, wherein the plurality of consecutive time domain sound level measurements is received from a sound sensor.

6. The environment controller of claim 4, wherein the generation of the N consecutive sets of frequency domain sound level measurements based on the plurality of consecutive time domain sound level measurements uses a Fast Fourier Transform algorithm.

7. The environment controller of claim 1, wherein the processing unit further determines N consecutive temperature measurements corresponding to the N consecutive sets of frequency domain sound level measurements and the inputs further include the N consecutive temperature measurements.

8. The environment controller of claim 7, wherein the determination of the N consecutive temperature measurements is based on data received via the at least one communication interface from a temperature sensor.

9. The environment controller of claim 1, wherein the processing unit further determines a temperature measurement corresponding to the N consecutive sets of frequency domain sound level measurements and the inputs further include the temperature measurement.

10. The environment controller of claim 9, wherein the determination of the temperature measurement is based on data received via the at least one communication interface from a temperature sensor.

11. The environment controller of claim 1, wherein the one or more output further comprises a predicted variation of carbon dioxide (CO2) level.

12. The environment controller of claim 11, wherein the processing unit further determines N consecutive CO2 level measurements corresponding to the N consecutive sets of frequency domain sound level measurements and the inputs further include the N consecutive CO2 level measurements.

13. The environment controller of claim 12, wherein the determination of the N consecutive CO2 level measurements is based on data received via the at least one communication interface from a CO2 sensor.

14. The environment controller of claim 11, wherein the processing unit further determines a CO2 level measurement corresponding to the N consecutive sets of frequency domain sound level measurements and the inputs further include the CO2 level measurement.

15. The environment controller of claim 14, wherein the determination of the CO2 level measurement is based on data received via the at least one communication interface from a CO2 sensor.

16. The environment controller of claim 1, wherein the processing unit further generates at least one command for controlling at least one controlled appliance and transmits the at least one command to the at least one controlled appliance via the at least one communication interface, the generation of the at least one command being based at least on the predicted variation of the temperature.

17. The environment controller of claim 16, wherein the at least one controlled appliance comprises a Variable Air Volume (VAV) appliance.

18. The environment controller of claim 1, wherein each sound level amplitude at the corresponding frequency consists of a sound pressure at the given frequency, a sound pressure level at the given frequency or a sound power at the given frequency.

19. The environment controller of claim 1, wherein the neural network inference engine implements a neural network comprising an input layer, followed by fully connected layers; the input layer comprising N*M neurons respectively receiving the sound level amplitudes; the predictive model comprising weights for the fully connected layers.

20. The environment controller of claim 1, wherein the neural network inference engine implements a neural network comprising one input layer, followed by at least one one-dimensional convolutional layer, followed by fully connected layers; the input layer comprising M neurons respectively receiving a one-dimension matrix, each one-dimension matrix comprising N sound level amplitudes at a given frequency among the M frequencies, the at least one one-dimensional convolutional layer applying a one-dimensional convolution to each one-dimension matrix; the predictive model comprising weights for the fully connected layers and parameters for the at least one one-dimensional convolutional layer.

21. The environment controller of claim 20, wherein the neural network further comprises at least one pooling layer.

22. The environment controller of claim 1, wherein the neural network inference engine implements a neural network comprising an input layer, followed by at least one two-dimensional convolutional layer, followed by fully connected layers; the input layer comprising one neuron receiving a two-dimensions matrix comprising the N*M sound level amplitudes, the at least one two-dimensional convolutional layer applying a two-dimensional convolution to the two-dimensions matrix; the predictive model comprising weights for the fully connected layers and parameters for the at least one two-dimensional convolutional layer.

23. The environment controller of claim 22, wherein the neural network further comprises at least one pooling layer.

24. A method for predicting temperature variations based on sound level measurements, the method comprising:
storing a predictive model in a memory of a computing device;
determining by a processing unit of the computing device N consecutive sets of frequency domain sound level measurements, each set of frequency domain sound level measurements comprising a given number M of sound level amplitudes at the corresponding given number M of frequencies, N and M being integers; and
executing by the processing unit of the computing device a neural network inference engine using the predictive model for inferring one or more output based on inputs, the inputs comprising the N consecutive sets of frequency domain sound level measurements, the one or more output comprising a predicted variation of a temperature.

25. The method of claim 24, wherein determining the N consecutive sets of frequency domain sound level measurements comprises receiving by the processing unit the N consecutive sets of frequency domain sound level measurements via a communication interface of the computing device.

26. The method of claim 25, wherein the N consecutive sets of frequency domain sound level measurements are received from a sound sensor.

27. The method of claim 24, wherein determining the N consecutive sets of frequency domain sound level measurements comprises:
receiving by the processing unit a plurality of consecutive time domain sound level measurements via a communication interface of the computing device; and
generating by the processing unit the N consecutive sets of frequency domain sound level measurements based on the plurality of consecutive time domain sound level measurements.

28. The method of claim 27, wherein the plurality of consecutive time domain sound level measurements is received from a sound sensor.

29. The method of claim 27, wherein the generation of the N consecutive sets of frequency domain sound level measurements based on the plurality of consecutive time domain sound level measurements uses a Fast Fourier Transform algorithm.

30. The method of claim 24, further comprising determining by the processing unit N consecutive temperature measurements corresponding to the N consecutive sets of frequency domain sound level measurements and the inputs further include the N consecutive temperature measurements.

31. The method of claim 30, wherein the determination of the N consecutive temperature measurements is based on data received via a communication interface of the computing device from a temperature sensor.

32. The method of claim 24, further comprising determining by the processing unit a temperature measurement corresponding to the N consecutive sets of frequency domain sound level measurements and the inputs further include the temperature measurement.

33. The method of claim 32, wherein the determination of the temperature measurement is based on data received via a communication interface of the computing device from a temperature sensor.

34. The method of claim 24, wherein the one or more output further comprises a predicted variation of carbon dioxide (CO2) level.

35. The method of claim 34, further comprising determining by the processing unit N consecutive CO2 level measurements corresponding to the N consecutive sets of frequency domain sound level measurements and the inputs further include the N consecutive CO2 level measurements.

36. The method of claim 35, wherein the determination of the N consecutive CO2 level measurements is based on data received via a communication interface of the computing device from a CO2 sensor.

37. The method of claim 34, further comprising determining by the processing unit a CO2 level measurement corresponding to the N consecutive sets of frequency domain sound level measurements and the inputs further include the CO2 level measurement.

38. The method of claim 37, wherein the determination of the CO2 level measurement is based on data received via a communication interface of the computing device from a CO2 sensor.

39. The method of claim 24, further comprising generating by the processing unit at least one command for controlling at least one controlled appliance and transmitting by the processing unit the at least one command to the at least one controlled appliance via a communication interface of the computing device, the generation of the at least one command being based at least on the predicted variation of the temperature.

40. The method of claim 39, wherein the at least one controlled appliance comprises a Variable Air Volume (VAV) appliance.

41. The method of claim 24, wherein each sound level amplitude at the corresponding frequency consists of a sound pressure at the given frequency, a sound pressure level at the given frequency or a sound power at the given frequency.

42. The method of claim 24, wherein the neural network inference engine implements a neural network comprising an input layer, followed by fully connected layers; the input layer comprising N*M neurons respectively receiving the sound level amplitudes; the predictive model comprising weights for the fully connected layers.

43. The method of claim 24, wherein the neural network inference engine implements a neural network comprising one input layer, followed by at least one one-dimensional convolutional layer, followed by fully connected layers; the input layer comprising M neurons respectively receiving a one-dimension matrix, each one-dimension matrix comprising N sound level amplitudes at a given frequency among the M frequencies, the at least one one-dimensional convolutional layer applying a one-dimensional convolution to each one-dimension matrix; the predictive model comprising weights for the fully connected layers and parameters for the at least one one-dimensional convolutional layer.

44. The method of claim 43, wherein the neural network further comprises at least one pooling layer.

45. The method of claim 24, wherein the neural network inference engine implements a neural network comprising an input layer, followed by at least one two-dimensional convolutional layer, followed by fully connected layers; the input layer comprising one neuron receiving a two-dimensions matrix comprising the N*M sound level amplitudes, the at least one two-dimensional convolutional layer applying a two-dimensional convolution to the two-dimensions matrix; the predictive model comprising weights for the fully connected layers and parameters for the at least one two-dimensional convolutional layer.

46. The method of claim 45, wherein the neural network further comprises at least one pooling layer.

47. A non-transitory computer program product comprising instructions executable by a processing unit of a computing device, the execution of the instructions by the processing unit of the computing device providing for predicting temperature variations based on sound level measurements by:
storing by the processing unit a predictive model in a memory of a computing device;
determining by the processing unit N consecutive sets of frequency domain sound level measurements, each set of frequency domain sound level measurements comprising a given number M of sound level amplitudes at the corresponding given number M of frequencies, N and M being integers; and
executing by the processing unit a neural network inference engine using the predictive model for inferring one or more output based on inputs, the inputs comprising the N consecutive sets of frequency domain sound level measurements, the one or more output comprising a predicted variation of a temperature.
